(19) United States
(12) Patent Application Publication
(10) Pub. No.: US 2013/0261666 A1
(43) Pub. Date: Oct. 3, 2013

(54) INTERSPINOUS FIXATION DEVICE

(71) Applicant: SPINESMITH PARTNERS, L.P., Austin, TX (US)

(72) Inventor: Mukund Gundanna, College Station, TX (US)

(73) Assignee: SpineSmith Partners, L.P., Austin, TX (US)

(21) Appl. No.: 13/851,736

(22) Filed: Mar. 27, 2013

Related U.S. Application Data
(60) Provisional application No. 61/616,645, filed on Mar. 28, 2012.

(51) Int. Cl. A61B 17/70

(52) U.S. Cl. CPC A61B 17/7068
USPC 606/248; 606/279

(57) ABSTRACT

A method of securing a portion of a spine is disclosed that includes preparing a first tunnel through a first spinous process and a second tunnel through a second spinous process of a spine, affixing one pair of complementary plates to each tunnel on each side of the spinous process, and affixing a rod into troughs in plates on one side of the spinous process.

### CROSS-REFERENCES TO RELATED APPLICATIONS

This Application claims the benefit under 35 U.S.C. §119(e) of U.S. Provisional Patent Application Ser. No. 61/616,645, filed Mar. 28, 2012, which is incorporated herein by reference in its entirety as if fully set forth herein.

### TECHNICAL FIELD OF THE INVENTION

The claimed invention relates to a device and process for interspinous fixation using complementary plates that affix to each other through a tunnel made in a spinous process, and using a rod that attaches to the plates.

### BACKGROUND OF THE INVENTION

Current devices and implants for fixation of spinous processes are sized and placed in the interspinous space. The current devices are affixed by clamping the devices into portions of the spinous process that is composed of fragile soft bone. The clamping thus can cause significant damage to the spinous process in these locations. The fixation onto the spinous processes is assumed, and may or may not be centered or fully purchased in the bone. With the angulation of the spinous process, it is hard to know how much purchase has been had in the bone based on looking at the remainder of the spinous process above the fixation device. It is also difficult to access the spinous process for anything else once the fixation device is clamped. Typically during removal of current implants, the spinous process will fracture and no revision instrumentation can be placed. It is not possible for currently available implants to be modular and allow for building or removing segments without significantly disrupting the retained segments. It is also not possible for currently available implants to compress the spine. Distraction is possible, but at significant risk to the integrity of the spinous process. If performed, arthrodesis and grafting is through a small barrel in the interspace. It is difficult to keep the graft in the interspace, and the volume of graft and exposure to cancellous bone is very limited. Most of the interspace is taken up by the implant. The current devices all play on similar design rationale to the well-known X-stop device, which is also placed in the interspinous space.

### SUMMARY OF THE INVENTION

In one broad respect, the invention is directed to a method of securing a portion of a spine, comprising: using a trial to determine the size of a projection-containing plate and a projection-receiving plate for a given spinous process, preparing a first tunnel through a first spinous process and a second tunnel through a second spinous process of a spine, providing at least two appropriate-sized projection-containing plates that each include (a) a projection that is adapted to be advanced through a spinous process tunnel, (b) a threaded trough on the side opposite to the projection wherein the trough is adapted to receive and attach to a rod, wherein the threaded trough includes a portion that is adapted to be broken off after the rod has been guided into the trough, and (c) spikes to perforate and grasp a spinous process, providing at least two appropriate-sized projection-receiving plates that (a) are connectable to a projection from a projection- containing plate and (b) include spikes to perforate and grasp a spinous process, inserting the projection of a projection-containing plate through the first tunnel, pressing the spikes of the projection-containing plate into the spinous process, and connecting the projection of the projection-containing plate to a projection-receiving plate that has been positioned on the opposite side of the first spinous process and that has been pressed so that the spikes of the another projection-receiving plate perforate and grasp the spinous process, inserting the projection of another projection-containing plate through the second tunnel, pressing the spikes of the projection-containing plate into the spinous process, and connecting the projection of the another projection-containing plate to another projection-receiving plate that has been positioned on the opposite side of the second spinous process and that has been pressed so that the spikes of the another projection-receiving plate perforate and grasp the spinous process, guiding a rod into the troughs of the projection-containing plates and connecting the rod to the projection-containing plates; and after guiding the rod into a trough breaking off the portions of the troughs that are adapted to be broken off.

In another broad respect, the invention is a method of securing a portion of a spine to a rod, comprising: preparing a tunnel through a spinous process of a spine, providing a projection-containing plates that includes (a) a projection that is adapted to be advanced through a spinous process tunnel, (b) a threaded trough on the side opposite to the projection wherein the trough is adapted to receive and attach to a rod, wherein the threaded trough includes a portion that is adapted to be broken off after the rod has been guided into the trough, and (c) spikes to perforate and grasp a spinous process, providing a projection-receiving plate that (a) is connectable to a projection from a projection-containing plate and (b) includes spikes to perforate and grasp a spinous process, inserting the projection of a projection-containing plate through the first tunnel, pressing the spikes of the projection-containing plate into the spinous process, and connecting the projection of the projection-containing plate to a projection-receiving plate that has been positioned on the opposite side of the spinous process and that has been pressed so that the spikes of the projection-receiving plate perforate and grasp the spinous process, guiding a rod into the trough of projection-containing plate and connecting the rod to the projection-containing plate; and after guiding the rod into a trough breaking off the portion of the troughs that is adapted to be broken off.

In another broad respect, the invention is an implant for spinous fixation, comprising: a first pair of complementary plates, comprising a first projection-containing plate and a first projection receiving plate, a second pair of complementary plates, comprising a second projection-containing plate and a second projection receiving plate, wherein the plates include spikes on the sides that include the projection member and projection-receiving member, and wherein one side of the projection-containing plate or the projection-receiving plate includes a trough for receiving and connecting to a rod adapted to be received in the troughs. This implant can be configured so that each trough includes a portion that is adapted to be broken off after guiding the rod into the trough.

### BRIEF DESCRIPTION OF THE DRAWINGS

FIG. 1 shows the positioning of trial plates on opposite sides of the spinous process in accordance with an embodiment of the invention;

FIG. 2 shows the preparation of a spinous process prior to installing a permanent plate in accordance with an embodiment of the invention;

FIG. 3 shows the positioning of a trial plate in accordance with an embodiment of the invention;

FIG. 4 shows the installation of permament plates onto a spinous process in accordance with an embodiment of the invention;

FIG. 5 shows a cross-sectional view of a spine where the permanent plates have been installed onto the spinous process in accordance with an embodiment of the invention; and

FIG. 6 shows the installation of permanent plates on spinous processes in accordance with an embodiment of the invention.

### DETAILED DESCRIPTION OF EXEMPLARY EMBODIMENTS

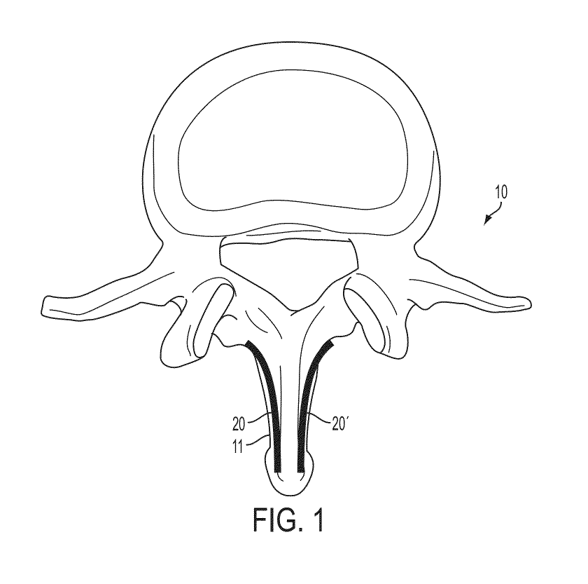

FIG. 1 illustrates a cross-sectional view of a spine 10 wherein two trial plates 20, 20′ are positioned on opposite sides of the spinous process 11. The trial plates can be parallel or flared. The trial plates can be inserted around the process without damaging the process. Also, the trial plates serve to identify the correct permanent plates based on the size and shape of the trial plates and the flare of the lamina.

Both the trial plates and permanent plates are sized and shaped to anatomically fit the process without torqueing the process. Both the trial plates and the permanent plates can come in a variety of sizes, e.g. small, medium, and large having different footprints to fit a given process. Each of these sizes can be parallel or flared. The variety of sizes and shapes is believed to provide a system with less inventory than current commercial systems.

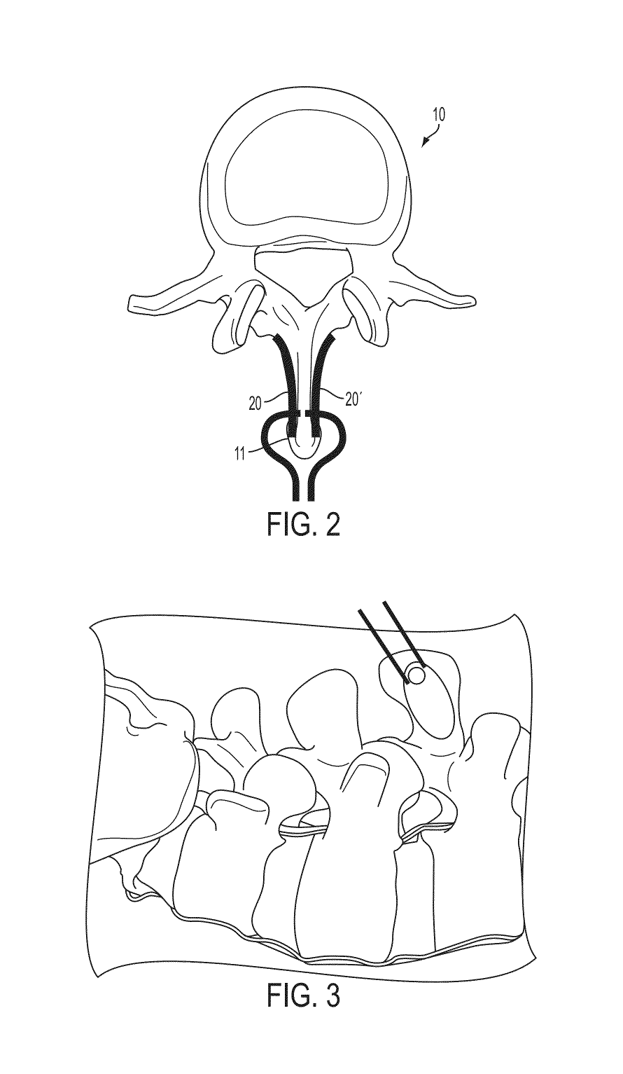

FIG. 2 illustrates the preparation of a spinous process 11 prior to installing a permanent plate. Thus, with trial plates in place (the plates include holes as illustrates in FIG. 3), a bone awl (like a towl clip) goes through the hole in one of the trial plates. The hole is started on the superficial side of the trial plate so that if the spinous process is cracked, the hole is in an area that will not be terribly harmful. Once the initial hole is made in the process, the hole (or, “tunnel”) is enlarged by sequential rasps. After the tunnel is made, the trial plates can be removed. The permanent plates can then be installed.

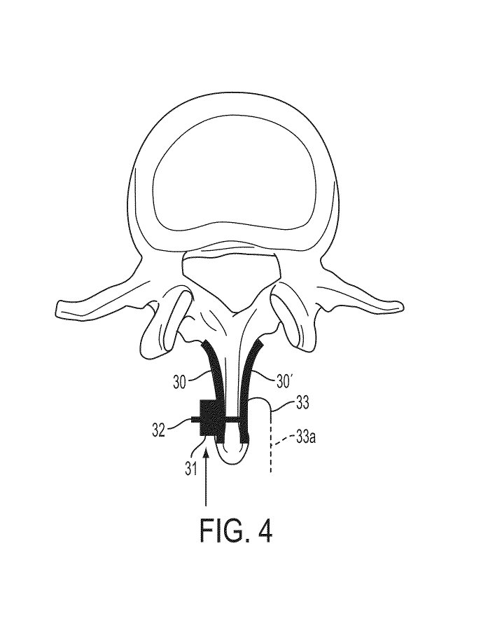

FIG. 4 illustrates permanent plates 30, 30′ (projection-receiving plate 30, projection-containing plate 30′) that have been installed onto a spinous process. The plates include a projection-containing plates for one side that includes (a) a projection that is adapted to be advanced through a spinous process tunnel, (b) a threaded trough on the side opposite to the projection wherein the trough is adapted to receive and attach to a rod, wherein the threaded trough includes a portion that is adapted to be broken off after the rod has been guided into the trough, and (c) spikes to perforate and grasp a spinous process. The complementary plate to the projection-containing plate is a projection-receiving plate that (a) is connectable to the projection from the projection-containing plate and (b) includes spikes to perforate and grasp a spinous process.

During use, the projection of a projection-containing plate is inserted through the first tunnel and the spikes of the projection-containing plate are pressed into the spinous process. The projection-receiving plate that is positioned on the opposite side of the spinous process so that the hole in the projection receiving plate aligns with the tunnel/projection. The projection-receiving plate is also pressed into the process so that the spikes of the projection-receiving plate perforate and grasp the spinous process. A suitable fastener 31 (or barrel) that fits the projection 32 is then employed to affix to the projection so that the two plates become connected and the spikes held in place so that the spikes do not back out. In general this process can be described as clamping the two sides (plates) onto the process so that a projection from one side goes through the bone tunnel and into the barrel (fastener) on the other side. A set screw, for example, that screws into the barrel locks the projection into place, thus connecting the two plates.

FIG. 4 also depicts a trough 33 on the projection-containing plate. The trough can optionally include a removable tab (33a section of trough 33) that helps guide a rod into place. Once the rod is in place the removable tab 33a can be broken off to reduce the size of the trough remaining in a body.

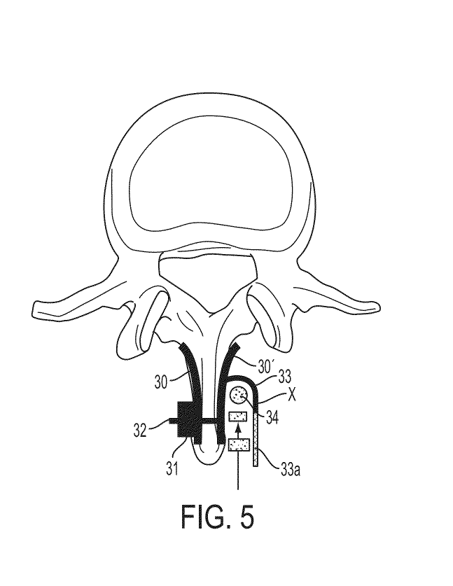

FIG. 5 shows a cross-sectional view of a spine where the permanent plates have been installed onto the spinous process. A rod 34 has been positioned in the trough, and a set screw, for example, is used to secure the rod in the trough. In this embodiment the trough 33 is suitably configured to include threads to secure the rod in place.

Alternatively the set screw can be screwed into a bore (not shown) on the side of the trough opposite the process, e.g. at point x in FIG. 5. Yet another alternative could be a barrel such as generally depicted in FIG. 5 for plate 30, where the additional barrel is channeled to receive the rod as well as to receive a set screw which screws into a bore to contact the rod and thus secure the rod in place.

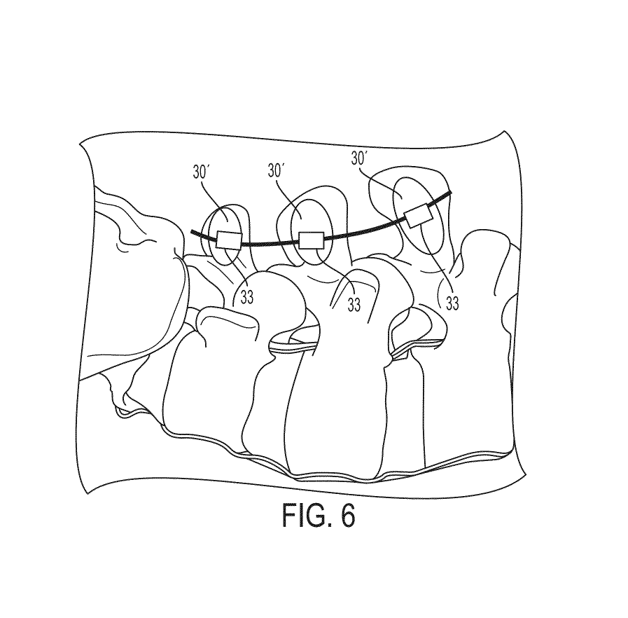

FIG. 6 illustrates a system of three sets of plates that have been installed in three spinous process, with a rod attached to the troughs 33 of the projection-containing plates 30′. In this embodiment the set screws engage the trough from above. Notably, the discs can be compressed or distracted prior to locking the rod in place.

In an alternative embodiment, the projection-receiving plates also include a trough, thus permitting two rods to be used, one rod on each side of the process. Such a configuration could permit greater strength of the system to hold the discs in a given configuration.

The surface of the plates that contacts the spinous process includes multiple spikes that are pressed into the process. The spikes are spread out across the face of the plate to help distribute stress across the process, as opposed to the stress being localized at the bone tunnel where the plates are attached together. The number of spikes can vary widely depending on their size, the size and condition of the spinous process, and other factors.

The system of this invention has a number of advantages. Importantly, the implant is fit to the spinous process rather than being positioned in the interspace between two spinous processes. In addition, greater bone contact (surface area) leads to less pressure on the bone and less bone fracture. Advantageously, the trial plates are used to size permanent plates and set the spinal area up for final placement of the permanent plates, as opposed to current systems. The inventive system permits a surgeon to easily compress, distract, and/or derotate the vertebral bodies. Advantageously, each spinous process is independently and segmentally plated, and then connected to other spinous processes with a rod that is cut to the appropriate length and contoured. This allows the user to build a construct to include as many spinal segments as desired. Furthermore, this feature allows future revisions of the construct—either adding, removing, or adjusting spinal segments at a later time—by simply removing the rod and then adding, removing, or adjusting the required plates, without having to remove or alter the plates that are in good position and are to be retained, and then reattaching the plates together with the appropriate rod. Importantly, because the implant does not fit in the interspace between two processes, the interspace is open, thus allowing a surgeon to readily place bone graft material in the interspace.

Further modifications and alternative embodiments of this invention will be apparent to those skilled in the art in view of this description. It will be recognized, therefore, that the present invention is not limited by these example arrangements. Accordingly, this description is to be construed as illustrative only and is for the purpose of teaching those skilled in the art the manner of canying out the invention. It is to be understood that the forms of the invention herein shown and described are to be taken as the presently preferred embodiments. Various changes may be made in the implementations and architectures. For example, equivalent elements may be substituted for those illustrated and described herein, and certain features of the invention may be utilized independently of the use of other features, all as would be apparent to one skilled in the art after having the benefit of this description of the invention.

What is claimed is:

1. A method of securing a portion of a spine, comprising:
using a trial plate to determine the size of a projection-containing plate and a projection-receiving plate for a given spinous process;
preparing a first tunnel through a first spinous process and a second tunnel through a second spinous process of a spine;
providing at least two appropriate-sized projection-containing plates that each include a projection that is adapted to be advanced through a spinous process tunnel, a threaded trough on the side opposite to the projection wherein the trough is adapted to receive and attach to a rod, wherein the threaded trough includes a portion that is adapted to be broken off after the rod has been guided into the trough, and spikes to perforate and grasp a spinous process;
providing at least two appropriate-sized projection-receiving plates that are connectable to a projection from a projection-containing plate and include spikes to perforate and grasp a spinous process;
inserting the projection of a projection-containing plate through the first tunnel, pressing the spikes of the projection-containing plate into the spinous process, and connecting the projection of the projection-containing plate to a projection-receiving plate that has been positioned on the opposite side of the first spinous process and that has been pressed so that the spikes of the another projection-receiving plate perforate and grasp the spinous process;
inserting the projection of another projection-containing plate through the second tunnel, pressing the spikes of the projection-containing plate into the spinous process, and connecting the projection of the another projection-containing plate to another projection-receiving plate that has been positioned on the opposite side of the second spinous process and that has been pressed so that the spikes of the another projection-receiving plate perforate and grasp the spinous process;
guiding a rod into the troughs of the projection-containing plates and connecting the rod to the projection-containing plates; and
breaking the portions of the troughs that are adapted to be broken off after the rod has been guided into the trough.

2. A method of securing a portion of a spine to a rod, comprising:
preparing a tunnel through a spinous process of a spine;
providing a projection-containing plates that includes (a) a projection that is adapted to be advanced through a spinous process tunnel, (b) a threaded trough on the side opposite to the projection wherein the trough is adapted to receive and attach to a rod, wherein the threaded trough includes a portion that is adapted to be broken off after the rod has been guided into the trough, and (c) spikes to perforate and grasp a spinous process;
providing a projection-receiving plate that (a) is connectable to a projection from a projection-containing plate and (b) includes spikes to perforate and grasp a spinous process;
inserting the projection of a projection-containing plate through the first tunnel, pressing the spikes of the projection-containing plate into the spinous process, and connecting the projection of the projection-containing plate to a projection-receiving plate that has been positioned on the opposite side of the spinous process and that has been pressed so that the spikes of the projection-receiving plate perforate and grasp the spinous process;
guiding a rod into the trough of projection-containing plate and connecting the rod to the projection-containing plate; and
breaking the portions of the troughs that are adapted to be broken off after the rod has been guided into the trough.

3. An implant for spinous fixation, comprising:
a first pair of complementary plates, comprising a first projection-containing plate and a first projection receiving plate,
a second pair of complementary plates, comprising a second projection-containing plate and a second projection receiving plate,
wherein the plates include spikes on the sides that include the projection member and projection-receiving member, and
wherein one side of the projection-containing plate or the projection-receiving plate includes a trough for receiving and connecting to a rod adapted to be received in the troughs.

4. The implant of claim 3 wherein each trough includes a portion that is adapted to be broken off after guiding the rod into the trough.
